(19) United States
(12) Patent Application Publication
(10) Pub. No.: US 2022/0293220 A1
(43) Pub. Date: Sep. 15, 2022

(54) Biological information inference apparatus and method utilizing biological species identification

(71) Applicant: HOMO MIMICUS CO., LTD, Daejeon (KR)

(72) Inventor: Sun Joong KIM, Daejeon (KR)

(21) Appl. No.: 17/625,067

(22) PCT Filed: Jul. 6, 2020
(86) PCT No.: PCT/KR2020/008776

§ 371 Date: Jan. 5, 2022

(30) Foreign Application Priority Data
Jul. 5, 2019 (KR) 10-2019-0081144
Jul. 6, 2020 (KR) 10-2020-0082524

(51) Int. Cl. G16B 50/00; G16B 45/00

(52) U.S. Cl. CPC G16B 50/00; G16B 45/00

(57) ABSTRACT

Disclosed are a biological information inference apparatus and method utilizing biological species identification. According to an embodiment of the present invention, provided is a biological information inference apparatus in which a biological species is recommended with respect to a user's query by a recommendation system, and at this time pieces of information stored in a biological system information causal model are utilized to infer biological information by combining factors having a connection with a biological species identification key.

### FIELD OF INVENTION

The present invention relates to biological information inference apparatus and method utilizing biological species identification.

### RELATED ART

Recently, an approach that enables a designer to extract or retrieve biological knowledge fast and precisely from explosively increasing documents in biology is required.

Through this approach, it will become possible to suggest a goal of effective development in various fields of technology such as developing a semi-permanent jointing method or IFF (identification of friend or foe) method for bio-inspired robot by use of biological knowledge.

But, the conventional retrieval algorithm for biological knowledge is remarkably poor at supporting a cognitive search process of designer.

Although bio information retrieval service accessible via Internet for providing comprehensive information related to organism such as gene sequence is partially implemented, it can provide information limited to biological relations of an organism but cannot provide an integrated retrieval for various information such as physical relations.

Besides, although a technique to extract relations between biological entities from biological structure documents by use of biological entity name, this technique is also based on information limited to biological relations of an organism.

As described above, conventional retrieval systems for biological knowledge can provide a keyword search for very narrow scope of information or a simple search result according to image matching.

In addition, scientific ways of utilizing the species identification key have not been proposed in the information system of biological system, and the species identification key has never been proposed or developed as an algorithm or information exchange device.

### SUMMARY

### Technical Objectives

In developing systems for reasoning biological or ecological characteristics of biological systems, an object of the present invention is to provide a system and method that helps to accurately capture the external or ecological characteristics of each organism by using a species identification key in the field of biology.

Other advantages and objectives will be easily appreciated through description below.

### Technical Solution

According to one aspect of the present invention, there is provided an apparatus of recommending a biological species in response to a user's question and utilizing information stored in a biological system information causal model to reason a biological information by combining elements having relationship with a species identification key.

There is provided a biological information reasoning apparatus utilizing a species identification, including an identification key collection unit configured for collecting a species identification key set, an identification key database configured for hierarchically managing a set of questions of the collected species identification key set, a recommendation system configured for recommending a biological system according to a user's question through a user terminal and a reasoning system configured for reasoning characteristics of the biological system from the set of questions of the collected species identification key set regarding a species corresponding to the biological system and a pre-built information system of biological system.

The species identification key set may consist of DAG graphs.

The species identification key set may include a logical node, which is a logical question that can be answered with yes or no and a chain of logical connections of the logical questions, wherein the species identification key set comprises one starting point and a plurality of endpoints as the logical nodes.

In the species identification key set, only one yes or no logical connection may be derived from each logical node, the logical node may receive connections from multiple logical connections, the chain connection of logical connections may be acyclic connection, and only one biological species may be assigned to one endpoint.

The identification key database may include a logical node table having a logical node unique number and a logical node text and a logical connection table having a biological species unique number and a logical connection graph of logical node numbers.

The reasoning system may reason a feature information of an organism with the logical node text of the logical node table, and the species unique number and the logical connection graph of the logical connection table.

Another aspect of the present invention, there is provided a biological information reasoning method being performed by a biological information reasoning apparatus utilizing a species identification, including transmitting a user question from a user terminal to a recommendation system, analyzing, by the recommendation system, the user question, driving a reasoning system when a relevant biological system information exists as a result of the analysis exists, searching for, by the reasoning system, a similar identification key from an identification key database, and linking to a biological system information database and recommending a related biological system.

In the case of linking to the biological system information database, a part and organ of the biological relationship according to a biological system information causal model of the biological system information database may have an additional connection relationship with the species identification key information, and an ecological behavior of the ecological relationship may have an additional connection with the species identification key information.

The identification key database may hierarchically manage a set of questions of the species identification key set, and the species identification key set may consist of DAG graphs.

The species identification key set may include a logical node, which is a logical question that can be answered with yes or no and a chain of logical connections of the logical questions, wherein the species identification key set comprises one starting point and a plurality of endpoints as the logical nodes.

In the species identification key set, only one yes or no logical connection may be derived from each logical node, the logical node may receive connections from multiple logical connections, the chain connection of logical connections may be acyclic connection, and only one biological species may be assigned to one endpoint.

The identification key database may include a logical node table having a logical node unique number and a logical node text and a logical connection table having a biological species unique number and a logical connection graph of logical node numbers.

The reasoning may utilize the logical node text of the logical node table and the species unique number and the logical connection graph of the logical connection table to reason a feature information of an organism.

Any other aspects, features, and advantages will be more clearly understood from the following detailed description taken in conjunction with the accompanying drawings and claims.

### Effects of Invention

According to embodiment of the present invention, in developing a system for reasoning the biological or ecological characteristics of a biological system, it is effective in helping to accurately capture the external or ecological characteristics of each organism by using the species identification key in the field of biology.

### BRIEF DESCRIPTION OF ACCOMPANYING DRAWINGS

FIG. 1 is a block diagram schematically showing a biological system information retrieval system according to one embodiment of the present invention;

FIG. 2 illustrates an ontology structure based on a causality for constructing a biological system information retrieval system according to one embodiment of the present invention;

FIG. 3 is a flowchart of reconfiguring a retrieval query according to one embodiment of the present invention;

FIG. 4 illustrates a similarity matrix and a sub-similarity matrix according to one embodiment of the present invention;

FIG. 5 illustrates a network graph that a causal model canvas unit schematizes according to one embodiment of the present invention;

FIG. 6 is a block diagram of an apparatus of reasoning biological system characteristics through species identification according to another embodiment of the present invention;

FIG. 7 is a flowchart of a method of reasoning biological system characteristics through species identification according to another embodiment of the present invention;

FIG. 8 is a classification diagram showing the biological species of soft trees; and

FIG. 9 is an exemplary diagram of a DAG graph.

### DETAILED DESCRIPTION

The invention can be modified in various forms and specific embodiments will be described below and illustrated with accompanying drawings. However, the embodiments are not intended to limit the invention, but it should be understood that the invention includes all modifications, equivalents, and replacements belonging to the concept and the technical scope of the invention.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded. Terms such as first, second, etc., may be used to refer to various elements, but, these element should not be limited due to these terms. These terms will be used to distinguish one element from another element.

Terms such as ˜ part, ˜ unit, ˜ module mean an element configured for performing a function or an operation. This can be implemented in hardware, software or combination thereof.

In describing the invention with reference to the accompanying drawings, like elements are referenced by like reference numerals or signs regardless of the drawing numbers and description thereof is not repeated. When it is determined that detailed description of known techniques involved in the invention makes the gist of the invention obscure, the detailed description thereof will not be made.

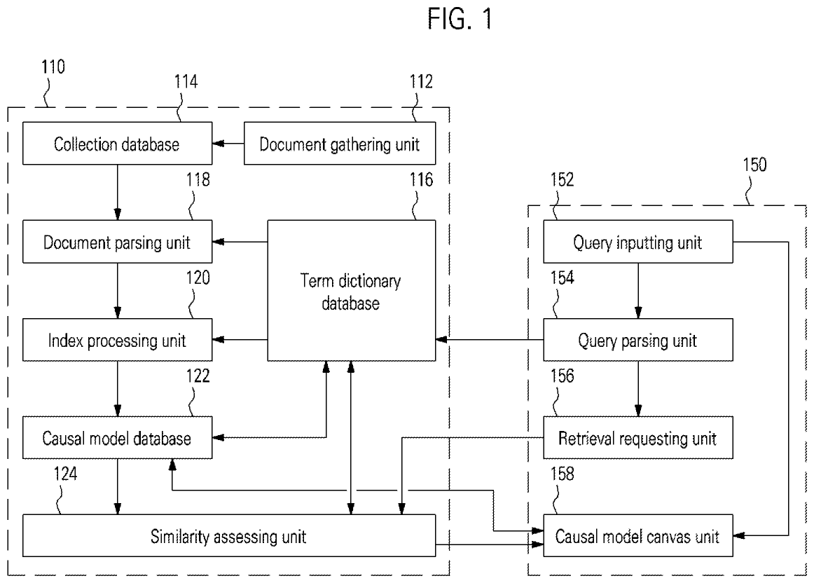

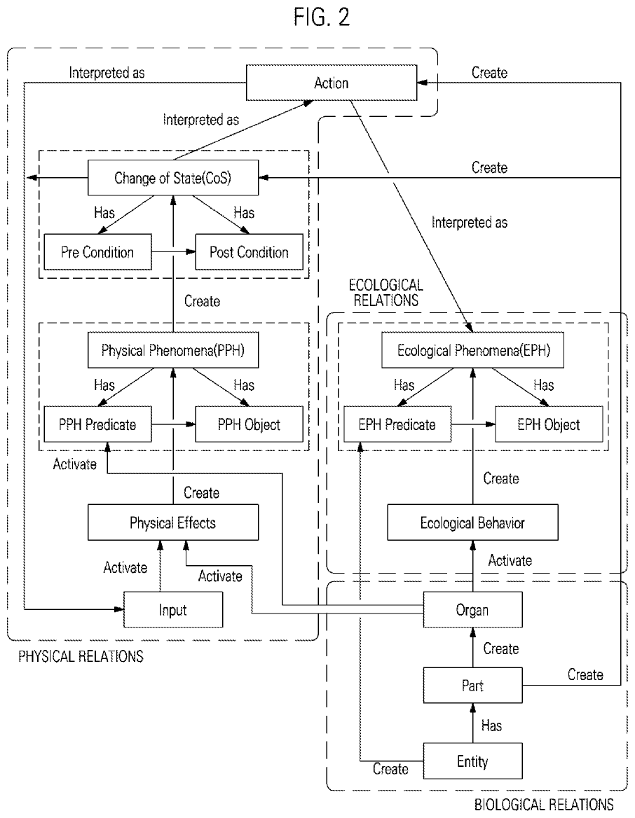

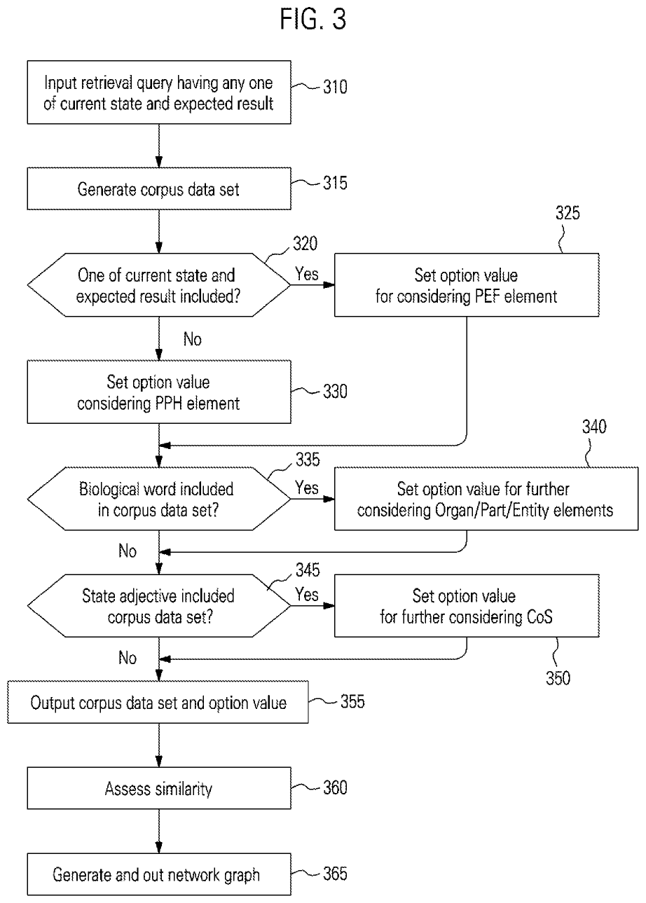

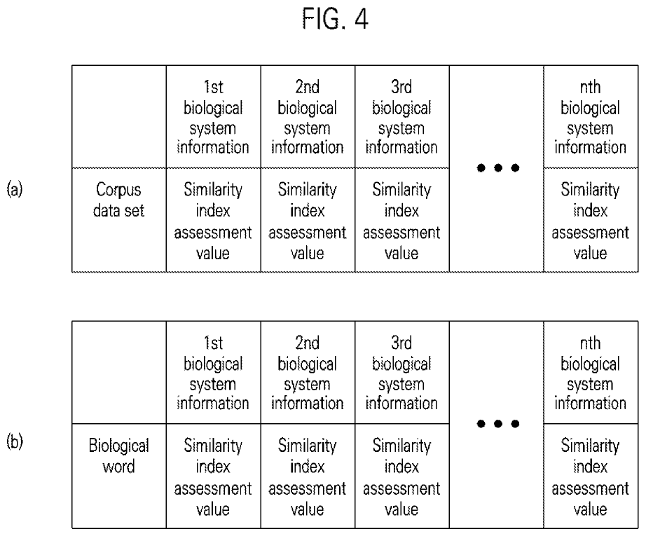

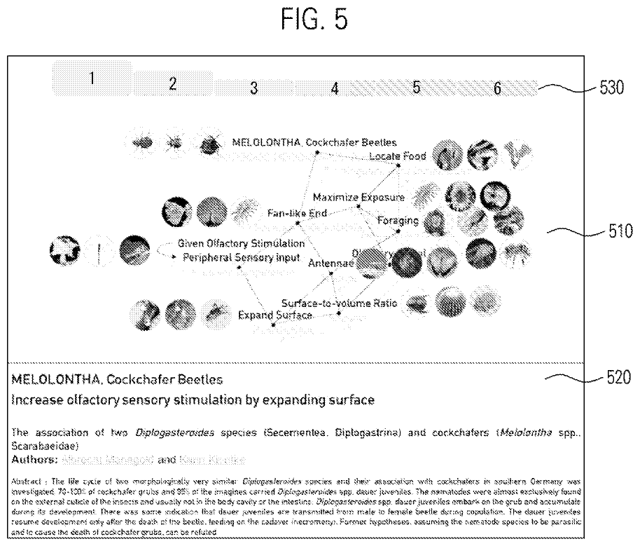

FIG. 1 is a block diagram schematically showing a biological system information retrieval system according to one embodiment of the present invention, FIG. 2 illustrates an ontology structure based on a causality for constructing a biological system information retrieval system according to one embodiment of the present invention, FIG. 3 is a flowchart of reconfiguring a retrieval query according to one embodiment of the present invention, FIG. 4 illustrates a similarity matrix and a sub-similarity matrix according to one embodiment of the present invention, and FIG. 5 illustrates a network graph that a causal model canvas unit schematizes according to one embodiment of the present invention.

Referring to FIG. 1, the biological system information retrieval system may include an information management device 110 and an information using device 150.

Although FIG. 1 illustrates that the information management device 110 and the information using device 150 are independent from each other and connected via wired or wireless communication, it will be appreciated that the information management device 110 and the information using device 150 can be integrated into one device if needed.

The information management device 110 is configured for constructing biological system information that can be a base for a bio-inspired design.

Biological system information specifies physical phenomena, biochemical phenomena and so on in an individual organism that is a subject of mimicking and application as physical relations, ecological relations, and biological relations. Biological system information can be extended an interaction between entities or an interaction between a plurality of species.

In other words, as it could be possible that one organism is directly mimicked but also possible that biological phenomena in organism, interaction(s) made by several entities, or interaction(s) made by various species of organisms are utilized directly or indirectly, biological system information can encompass biological phenomena in individual organism or interactions between organisms or species in order for designers to conceive various ideas in wider range.

For example, if biological information about European-starling having an enzyme that can catalyze alcohol decomposition for alcohol detoxification is stored and managed, a designer who is trying to develop a product for catalyzing alcohol decomposition can access the information management device 110 by using the information using device 150, and can retrieve and utilize information about European-starling by searching biological system information about catalyzing alcohol decomposition.

The information management device 110 may include a document gathering unit 112, a collection database 114, a term dictionary database 116, a document parsing unit 118, an index processing unit 120, a causal model database 122, and a similarity assessing unit 124.

The document gathering unit 112 collects BS (Biological structure) documents constituting of natural language. BS documents may be, for example, natural-language based HTML document arranged by biologists. Of course, author or type of BS documents should not be limited to the aforementioned, but any documents available for categorizing physical relations, ecological relations, and/or biological relations, and creating a causal model would be enough.

The collection database 114 stores BS documents that the document gathering unit 112 collected.

In the term dictionary database 116, terms that are needed to index physical relations, ecological relations, and/or biological relations, which are included in biological system information.

In the term dictionary database 116, a scientific name dictionary in which scientific name terms, for example, according to ITIS (International Taxonomy Information Systems) standard, references quoted in STONE's paper publically published in 2014, and so on may be included as index terms. By using scientific name terms, it would be advantageous for the present invention that can collect biological system information about 21,000 genera based on ITIS.

In addition, since function, material, energy, and/or signal terms are needed to index physical relations and ecological relations respectively, a function term dictionary, a material term dictionary (e.g., Material>Liquid>acid, chemical, water, blood, etc.), energy term dictionary (e.g., Energy>Hydralic>pressure, osmosis etc.), and/or signal term dictionary (e.g., Signal>Sense>Detect>detect, locate, see/Signal>Status>change, fatty, variation, etc.), which are edited by experts, may be stored in the term dictionary database 116. Terms related to EPH (Ecological Phenomena) may be composed of data defining classification relation according to each category of function, material, energy, and/or signal.

The document parsing unit 118 parses BS documents collected by the document gathering unit 112 to analyze a sentence structure of BS documents and to construct the sentence as a tree. The document parsing unit 118 may use a Scrapy parsing unit.

The index processing unit 120 indexes information that the document parsing unit 118 analyzed according to an ontology structure (see FIG. 2) which represents a biological system based on causality in which the conventional SAPPhIRE model is complemented.

That is, for information analyzed by the document parsing unit 118, the index processing unit 120 indexes biological relations of individual organism based on scientific name terms stored in the term dictionary database 116, and indexes each of physical relations and ecological relations among biological system of the organism based on terms representing function, material, energy, and/or signal respectively.

Biological system information may be derived from a triple form of subject-predicate-object, but, as shown in FIG. 2, may be structured to combine physical relations, ecological relations and/or biological relations that represent a mechanism of the organism and a causality manifested through the mechanism.

The smallest unit for indexing organism based on information analyzed from the collected BS documents is node, and connection information of each node forms relationship information.

Referring to FIG. 2, in physical relations of biological system information, Input (e.g., energy, signal, and/or material input) may activate PEF (Physical Effects), PEF may create PPH (Physical Phenomena), PPH may create CoS (Change of State), and CoS may be interpreted as Action.

Physical relations is information representing in a causality way that one organism undergoes a certain CoS and what causes a certain PPH through a certain PEF to achieve a certain objective (Action, Goal).

In detail, CoS relates to how a state is changed between before achieving the objective and final result, and a static state of pre condition and post condition may be indexed in a dynamic relationship.

PEF relates to a strategy used to achieve the objective, and may be generally indexed as strategies that are contained in an ecology dictionary, a physics dictionary, etc., to have definitions (i.e., definition corresponding to the word).

PPH relates to how a strategy is specifically implemented, and may be indexed with a combination of verb and object that are terms defined in a function term dictionary (verb), and an energy dictionary, a material dictionary, and/or signal dictionary (noun as object), which were edited by experts to represent how it is specifically implemented.

For example, if the European starling detoxifies alcohol, an alcohol detoxification may correspond to Action, CoS may be a change from high concentration of alcohol to low concentration of alcohol, and an alcoholism treatment may be PEF. Thus, Action, that is, objective can be achieved with an alcohol decomposition as PPH.

Specifically, Input such as ‘many alcohol molecules’ may activate PEF such as ‘alcoholism treatment’, ‘alcoholism treatment’ may create PPH such as ‘catalyzing alcohol decomposition’, as ‘catalyzing alcohol decomposition’ may create CoS causing ‘high concentration of alcohol’ (i.e., pre condition) to be changed to ‘low concentration of alcohol’ (i.e, post condition), and finally this CoS may be interpreted as Action such as ‘alcohol detoxification’. Also from an analytical point of view, Action of ‘alcohol detoxification’ may be reinterpreted as a cause such as Input of ‘many alcohol molecules’.

In addition, Action may be interpreted as EPH, so Action can be understood as a physical ‘strategy’ that an organism will take to do a certain behavior (or habit). For example, if a designer who wants to develop an alcohol addiction treatment becomes aware of an ecological relation such that European starlings are likely to eat fermented fruits containing alcohol, the designer may infer an ecological relation of alcoholic who needs alcohol detoxification from the ecological relation of European starling, and thus may apply Action of ‘alcohol detoxification’ that European starling takes as physical strategy to do the behavior (habit) to develop alcoholic treatment as design strategy.

In exemplary case that the collected BS documents contain information about European starling capable of detoxifying alcohol, structured biological system information stored in the term dictionary database 116 can be shown in Table 1 as example. Of course, it will be appreciated that terms stored in correspondence with each node (i.e., Input, PEF, etc.) may be increased and diversified, if European starling has various characteristics.

TABLE 1

| | | |
|---|---|---|
| Input | <Alcoholic compound> | <Alcohol> |
| Physical Effects | <Alcoholism treatment> | <Alcoholism-treatment> |
| Physical | <Catalyze> + <Alcohol | <Catalyze> + |
| Phenomena | decomposition> | <Alcohol + Decomposition> |
| Change of State | <High concentration of alcohol> + | <High + Density + of + Alcohol> + |
| | <Low concentration of alcohol> | <Low + Density + of + Alcohol> |
| Action | <Alcohol detoxification> | <Alcohol + Detoxification> |
| Ecological Phenomena | <Ingest> + <Fermented fruit> | <Ingest> + <Fermented + Fruit> |
| Ecological Behaviors | <Alcohol abuse> | <Alcohol + Abuse> |
| Organ | <Alcohol decomposition enzyme> | <Enzyme> |
| Part | <Stomach> | <Stomach> |
| Entity | <European starling> | <European-starling> + <Sturnus vulgaris> |

In addition, in an exemplary cast that another collected BS documents contain information about European starling having a light skeletal system for reducing air resistance, biological system information about European starling may be additionally generated and managed as shown in Table 2.

TABLE 2

| | | |
|---|---|---|
| Input | <Kinetic energy> + <Air resistance> | <Kinetic + Energy> + <Air> |
| Physical Effects | <Light weight skeletal system> | <Light-skeletal-system> |
| Physical Phenomena | <Reduce> + <Mass> | <Reduce> + <Body + Weight> |
| Change of State | <High weight> + <Low weight> | <High + Weight> + <Low + Weight> |
| Action | <Reducing energy consumption> | <Reduce + Energy + Consumption> |
| Ecological Phenomena | <Increase> + <Flight time> | <Increase> + <Flight + Time> |
| Ecological Behaviors | <Flight> | <Flying> |
| Organ | <Bone> | <Bone> |
| Part | <Skeletal system> | <Skeletal-system> |
| Entity | <European starling> | <European-starling> + <Sturnus vulgaris> |

Referring to Table 2, biological system information about European starling is that Inputs such as kinetic energy and air resistance may activate PEF such as a light skeletal system, the light skeletal system may generate PPH such as a reduction in bone weight, the reduction in bone weight may create CoS causing a heavy mass to be changed to a light mass, and finally this CoS may be interpreted as Action such as a reduction in energy consumption. Also from an analytical point of view, Action of reduction in energy consumption may be reinterpreted as a cause such as Input of high kinetic energy and air resistance.

In addition, in ecological relations that European starling has a habit of flying efficiently, the designer may regard an ecology of European starling as an ecology of flying object (i.e., in flight), and may apply Action of reduction in energy consumption as a physical strategy that European starling takes to do the behavior to a design strategy for developing a flying object.

As can be seen in FIG. 2 and Tables 1 and 2 respectively, biological relations of biological system information may be consist of Organ, Part, and Entity. Biological relations may indicate that biological phenomena are associated with which organ of an organ in an organism, and Part refers to a part to which the organ belongs.

Entity is an element for indexing that each biological system information is associated with which organism, is the owner of Organ and Part, and is the organism in which the biological phenomena can be observed.

For example, in case of a beetle that produces iridescent color, the beetle may be indexed as Entity, shell may be indexed as Part of biological system since cuticle belongs to shell of beetle, and cuticle of shell may be indexed as Organ.

Referring again FIG. 1, the ontology structure (see FIG. 2) designated by the index processing unit 120 and biological system information that is generated based on terms from each dictionary stored in the term dictionary database 116 are stored in the causal model database 122. Thumbnail images corresponding to each biological system information may be further stored in the causal model database 122.

Hereinafter, a syntax for storing terms for each element in the causal model database 122 will be briefly described.

CoS element may be stored according to a syntax of Expression 1.

COS_{Biological System}={Stateₚᵣₑ,Stateₚₒₛₜ}

Stateₚᵣₑ={Adjₚᵣₑ,Nounₚᵣₑ}

Stateₚₒₛₜ={Adjₚₒₛₜ,Nounₚₒₛₜ} Expression 1

CoS may be stored as a pre condition Stateₚᵣₑ and a post condition, Stateₚₒₛₜ, and consist of an adjective part Adj and a noun part Noun. In the term dictionary database 116, the adjective part index terms may be stored in a state adjective dictionary, and the noun part index terms may be stored in the material term dictionary, the energy term dictionary, and/or the signal term dictionary respectively.

PPH element may be stored according to a syntax of Expression 2.

PPH_{Biological System}={Predicate_{physical},Object_{physical}} Expression 2

PPH may consist of a verb part Predicate_{physical} and a noun part(Object_{physical} In the term dictionary database 116, the verb part index terms may be stored in the function term dictionary, and the noun part index terms may be stored in the material term dictionary, the energy term dictionary, and/or the signal term dictionary respectively, as described above.

PEF element may be stored according to a syntax of Expression 3.

PEF_{Biological System}={Index_{physicaleffect}} Expression 3

PEF may be indexed with one of index terms registered in a PEF index term dictionary stored in the term dictionary database 116. The PEF index term dictionary may be stored in a format of ‘index term’ and ‘definition of index term’ (e.g., ‘Camouflage’+‘Definition of camouflage’) in the term dictionary database 116.

Input element may be stored according to a syntax of Expression 4.

INP_{Biological System}={Indexₘₐₜₑᵣᵢₐₗ,Index_{energy},Index_{signal}} Expression 4

Input that activates biological system information may consist of related material index term Indexₘₐₜₑᵣᵢₐₗ, energy index term Index_{energy}, and/or signal index term Index_{signal}. These may be designated from terms registered in the material term dictionary, the energy term dictionary, and/or signal term dictionary stored in the term dictionary database 116.

EPH element may be stored according to a syntax of Expression 5.

EPH_{Biological System}={Predicate_{ecological},Object_{ecological}} Expression 5

EPH may consist of a verb part Predicate about ‘How’ and a noun part Object about ‘What’. For example, biological phenomena (camouflage) that cause an illusion to prevent from being detected by an enemy may have a biological function of avoiding enemy (body-material). Index terms of the verb part and the noun part may be stored in advance stored in the term dictionary database 116 as the function term dictionary, the material term dictionary, the energy term dictionary, and/or signal term dictionary.

EBH (Ecological Behavior) element may be stored according to a syntax of Expression 6.

EBH_{Biological System}={Index_{ecologicaleffect}} Expression 6

EBH element may be indexed with one of index terms registered in EBH index term dictionary stored in the term dictionary database 116. For example, the biological phenomena that cause an illusion of foe to prevent from being detected has a biological behavior such as camouflage. Index term dictionary may be stored in a format of ‘index term’ and ‘definition of index term’ (e.g., ‘Herbivore’+‘Definition of herbivore’) in the term dictionary database 116.

Organ element and Part element may be stored respectively according to a syntax of Expression 7.

ORG_{Biological System}={String_{organ}}

PRT_{Biological System}={Stringₚₐᵣₜ} Expression 7

Organ element and Part element may be indexed with terms in a biological word dictionary stored in the term dictionary database 116.

Entity element for indexing that biological system information is associated with which organism may be stored according to a syntax of Expression 8.

ENT_{Biological System}={ID_{ITIS},Index_{scientificname},Index_{commonname}} Expression 8

In order to make an association search possible, Entity may be indexed by a scientific name according to ITIS system, and ID_{ITIS} may index unique ID (number) of organism, Index_{scientificname} may index scientific name (text), and Index_{commonname} may index a common name (text) from ‘ITIS scientific name dictionary’. ITIS scientific name dictionary for indexation may be stored in the term dictionary database 116.

Action element may be stored respectively according to a syntax of Expression 9.

ACT_{Biological System}={String_{action}} Expression 9

Action element may be not stored in a dictionary, and indexed with a description summarized from a design strategy that a designer can obtain from biological system information.

As described above, since biological system information may be represented and respectively indexed with causal model in which physical relations, ecological relations and/or biological relations in each organism have mutual connections (directionality), it is advantageous for a designer to retrieve biological system information that is useful to his idea.

The similarity assessing unit 124 may receive the retrieval query from the retrieval requesting unit 156, may assess similarity between the retrieval query and each biological system information stored in the causal model database 122, and may provide biological system information having similarity equal to or greater than a threshold value to the causal model canvas unit 158. The similarity assessing unit 124 may manage biological system information store in the causal model database 122, for example, in Python language.

Detailed operations of the similarity assessing unit 124 will be described in connection with the retrieval requesting unit 156 and the causal model canvas unit 158 of the information using device 150.

The information using device 150 is configured for retrieving biological system information stored in the information management device 110 and receiving retrieval result, and may include a query inputting unit 152, a query parsing unit 154, a retrieval requesting unit 156, and the causal model canvas unit 158.

The query inputting unit 152 is a means for retrieving biological system information to which a designer may input a retrieval query corresponding to his needs (see 310 in FIG. 3).

The retrieval query may be in various forms including at least one word such as phrase, sentence, or paragraph.

However, in the present embodiment, the retrieval query may consist of natural-language phrase, and may be, for example, described by a combination of <Current state> and <Expected result>.

For this, although it is possible that the query inputting unit 152 may allow to describe <Current state> and <Expected result> in natural-language phrase in one query input slot (e.g., input window for search words), it is also possible to implement such that the query inputting unit 152 may provide the first query input slot for inputting <Current state> in natural-language phrase and the second query input slot for inputting <Expected result> in natural-language phrase, respectively.

In case that the retrieval query is described in a combination of <Current state> and <Expected result>, it will be advantageous that the causality will be more clarified when conducting retrieval, and it will be also effective because biological system information according to the present embodiment adopts a causal model that is expressed in a homogeneous structure.

The query parsing unit 154 may decompose the query phrase inputted by a designer with use of the query inputting unit 152 into tokens, which are words at meaningful level that are processed by a conventional natural language processing method, and analyze grammatical components of each token (e.g., adjective, verb, noun, etc.). In addition, the query parsing unit 154 may refer to terms stored in the term dictionary database 116 of the information management device 110 to generate corpus data set of tokens with query phrases of <Current state> and <Expected result> (See 315 in FIG. 3).

For example, if a designer may input ‘the blood alcohol level is very high’ as retrieval query for <Current state> and ‘the blood alcohol level is normal’ as retrieval query for <Expected result> in order to obtain an idea for developing alcoholism treatment, the query parsing unit 154 may generate [blood, alcohol, level, very, high] as a corpus data set for <Current state> and [blood, alcohol, level, normal] as a corpus data set for <Expected result>.

As described above, the corpus data set may be represented in form of a list by tokenizing words and eliminating sentence symbols and stopwords (e.g., a, an, for, and, etc.).

The retrieval requesting unit 156 may confirm whether the corpus data set that is inputted by use of the query inputting unit 152 and analyzed by the query parsing unit 154 exists for <Current state> and <Expected result>, and provide corpus data set to which a corresponding option value is added to the similarity assessing unit 124.

Even in case that corpus data set corresponding to the retrieval query to be provided to the similarity assessing unit 124 includes any one of <Current state> and <Expected result>, the similarity assessing unit 124 may be implemented to conduct a retrieval of biological system information and perform a similarity decision. Of course, in case that no corpus data set for <Current state> and <Expected result> exists, this means that no retrieval query is inputted so the following retrieval process will not be performed.

This is because that the bio-inspired design basically assumes a design thinking based on an inference strategy. Thus, a designer may not specify <Expected result> for the purpose of checking various results available under <Current state> condition in order to find idea out, and also may not specify <Current state> for the purpose of checking various pre conditions available under <Expected result>.

Namely, not specifying any one of <Current state> and <Expected result> may be interpreted as an intention not to put a limitation on thinking, and this is a way of design thinking that help a designer infer inventively.

For example, in case that a causality is specifically fixed by associating <Current state> of ‘high concentration of alcohol’ with <Expected result> of ‘low concentration of alcohol’, result such as ‘high concentration of alcohol’ is maintained, but biological system information about Pelotomaculum thermopropionicum that uses alcohol as an energy source cannot be retrieved.

The operation of the retrieval requesting unit 156 will be described in detail. In case that any one of <Current state> and <Expected result> is specified in the retrieval query, the retrieval requesting unit 156 may utilize PEF element that represents CoS most abstractly among ontology structure of biological system information to set an option value that allows the similarity assessing unit 124 to assess a similarity between information indexed with PEF element of biological system informations stored in the causal model database 122 and corpus data set of the retrieval query, also to draw a similarity matrix, and to provide biological system informations that are equal to or greater than a threshold value to the causal model canvas unit 158 (See 320 and 325 in FIG. 3).

However, in case that both <Current state> and <Expected result> are described in the retrieval query, the retrieval requesting unit 156 may utilize PPH among ontology structure of biological system information to set an option value that allows the similarity assessing unit 124 to assess a similarity between information indexed with PPH element of biological system informations stored in the causal model database 122 and corpus data set of the retrieval query, also to draw a similarity matrix, and to provide biological system informations that are equal to or greater than a threshold value to the causal model canvas unit 158 (See 320 and 330 in FIG. 3).

In detail, since <Expected result> shows an expected behavior as result of change, <Expected result> may allow the similarity assessing unit 124 to collect verb tokes from corpus data set of <Expected result> and to assess similarity between each information indexed as PPH elements. On the other hand, since <Current state> shows a target to be changed, <Current state> may allow the allow the similarity assessing unit 124 to collect noun tokes from corpus data set of <Expected result> and to assess similarity between each information indexed as PPH elements.

By aggregating the calculation result of the similarity assessing unit 124 for verb tokens of <Expected result> and the calculation result of the similarity assessing unit 124 for noun tokens of <Current state>, the similarity assessing unit 124 may draw the similarity matrix and provide biological system informations that are equal to or greater than the threshold value to the causal model canvas unit 158.

In addition, if a term registered in the biological word dictionary stored in the retrieval requesting unit 156 is found, the retrieval requesting unit 156 may set an option value that allows the similarity assessing unit 124 to further consider Organ, Part and Entity elements among the ontology structure of biological system information when assessing the similarity and also to use similarity assessment result when drawing the similarity matrix (See 335 and 340 in FIG. 3).

Here, a biological word relates to an organ, a part and/or an entity name (e.g., common name, scientific name, etc.) of an organism such as sensory-organ, lung, European starling.

But, if a term registered in the biological word dictionary stored is not found, the retrieval requesting unit 156 may set an option value that allows the similarity assessing unit 124 not to consider Organ, Part and Entity elements when assessing the similarity.

In addition, if a term registered in the state adjective dictionary stored in the retrieval requesting unit 156 is found, the retrieval requesting unit 156 may set an option value that allows the similarity assessing unit 124 to further consider CoS elements among the ontology structure of biological system information when assessing the similarity and also to use similarity assessment result when drawing the similarity matrix (See 345 and 350 in FIG. 3)

Here, a state adjective relates to size, shape, state, color, age, material, etc., among adjectives such as high, small, enormous, round, ceramic, metal, and so on. But, if a term registered in the state adjective dictionary stored is not found, the retrieval requesting unit 156 may set an option value that allows the similarity assessing unit 124 not to consider CoS element when assessing the similarity.

The retrieval requesting unit 156 may provide the similarity assessing unit 124 with corpus data set and option value that are generated according to the inputted retrieval query to request the retrieval (See 355 in FIG. 3).

The causal model canvas unit 158 may measure a derivativity (i.e., interrelationship) between at least one biological system information provided as a similarity index assessment result from the similarity assessing unit 124, and generate a network graph (see FIG. 5) by use of the measure derivativity (See 355 and 360 in FIG. 3). Of course, it will be appreciated that the similarity assessing unit 124 may measure the derivativity and the causal model canvas unit 158 may generate the network graph by use of the result of derivativity measurement.

Hereinafter, it will be described that the similarity assessing unit 124 conducts the retrieval by use of corpus data set corresponding to the retrieval query that the retrieval requesting unit 156 provides and biological system information about each organism stored in the causal model database 122, and assesses the similarity (See 355 in FIG. 3).

In order to perform the similarity assessment on biological system information stored in the causal model database 122 and corpus data set of <Current state> and/or <Expected result>, in case that n biological system informations are stored in the causal model database 122, the similarity assessing unit 124 may generate a 1×n similarity matrix for comparison with corpus data set (See (a) in FIG. 4). Each similarity index assessment value may be initialized to zero before performing the similarity assessment.

In case that corpus data set for any one of <Current state> and <Expected result> corresponding to the retrieval query is provided, the similarity assessing unit 124 may calculate a degree of topic interrelationship between the corpus data set and definition text (stored in PEF index term dictionary) for each of n biological system informations stored in the causal model database 122 by use of tf-idf (Term Frequency-Inverse Document Frequency) scheme) store the calculated value as similarity index assessment values of each biological information. If similarity value that was calculated already in previous similarity index assessment process exists, they will be summed.

Tf-idf scheme is a conventional scheme for comparing similarity between two documents with similarity of terms (tokens) used in each document. For example, in case that corpus data consists of [blood, alcohol, level, very, high], Tf-idf scheme compares the number of appearances in documents about ‘Alcoholism-treatment’ that is definition text of index term of PEF element with the number of appearances in definition documents for all terms in PEF index term dictionary. Since tokens such as level, very, high, etc. are frequently used in most of documents, it will be appreciated that relatively low similarity index value may be assigned to these tokens compared to other tokens such as blood, alcohol, etc.

But, in case that only corpus data set for both <Current state> and <Expected result> is provided, the similarity assessing unit 124 may generate a verb token set Wp by extracting verb tokens from corpus data set W_{ER} of <Expected result> and a noun token set Wo by extracting noun tokens from corpus data set W_{CS} of <Current state> by use of a conventional POST (Part of speech tagging) algorithm and so on. For example, in case that corpus data set of <Current state> consists of [blood, alcohol, level, very, high], since there is no token that can be considered as verb, verb token set Wp will be empty, but the noun token set Wo will be generated as [blood, alcohol, level].

The similarity assessing unit 124 may generate the first similarity index calculation value by calculating similarity index between terms in the verb token set and verb part (Predicate_{physical}, see Expression 2) of PPH element of each biological system information. In addition, the similarity assessing unit 124 may generate the second similarity index calculation value by calculating similarity index between terms in the noun token set and noun part (Object_{physical}) of PPH element of each biological system information, and store multiplication of the first and the second similarity index calculation values as similarity index assessment value for each biological system information. If similarity index assessment value that was calculated already in previous similarity index assessment process (e.g., similarity index assessment based on the presence/absence of biological word) exists, they will be summed.

In above example, since the verb token set Wp is empty, the first similarity index calculation value will be zero. But, in case that the verb token set Wp is not empty and PPH element of a certain biological system information is indexed as <Adjust>+<Direction+of+Incident+Light>, the similarity index between the verb token in the verb token set Wp and <Adjust> as verb part of PPH element will be calculated.

As described above, since verb terms are registered in the function term dictionary stored in the causal model database 122, the first similarity index calculation value may be generated by calculating a semantic distance between the verb token of the verb token set Wp and Adjust.

The function term dictionary may be composed of a tree data structure to calculate semantic distance between each term. The first similarity index calculation value may be generated as a distance that verb token reaches Adjust via a parent node that is common and nearest from both verb token and Adjust (i.e., the number of edges connecting each hierarchical node). Thus, as farther the nearest parent node is away from the highest node, as higher the first similarity value will be. This type of tree data structure may be structured in similar manner to a hierarchical structure having a connection relationship between nodes so as to calculate the degree of kinship.

In addition, in case that the noun token set Wo is not empty and PPH element of a certain biological system information is indexed as <Adjust>+<Direction+of+Incident+Light>, similarity index between noun token of the noun token set Wo and ‘Direction’ and ‘Light’ as noun part of PPH element may be calculated. The second similarity index calculation value may also be calculated by use of semantic distance of term in same manner as the process of the first similarity index calculation value, and if nouns to be calculated are plural (e.g., ‘Direction’ and ‘Light’), for example, an average, a sum, or a maximum value of these may be calculated as the second similarity index calculation value.

Then, the similarity assessing unit 124 may check whether the state adjective (e.g., small, high, etc.) exists in corpus data set corresponding to the retrieval query, and if exists, further perform similarity index assessment in consideration with the state adjective.

In case that the state adjective is found in corpus data set of <Current state> and/or <Expected result>, all multiplication of frequencies found in adjective part (See Expression 1) among index information of CoS element of each biological system information stored in the causal model database 122 may be stored as similarity index assessment value for each biological system information. If similarity index assessment value that was calculated already in previous similarity index assessment process exists, they will be summed. The state adjective of corpus data set of <Current state> may be compared to adjective part Adjₚᵣₑ of pre condition and the state adjective of corpus data set of <Expected result> may be compared to adjective part Adjₚₒₛₜ of post condition, and if an adjective is found both in corpus data sets of <Current state> and <Expected result>, a multiplication of each frequency may be stored as the similarity index assessment value.

For example, in case that CoS element of a certain biological system information consists of <High+Weight> and <Low+Weight>, the adjective part of pre condition is ‘High’ and the adjective part of post condition is ‘Low’. Assuming that state adjective of corpus data set of <Current state> is ‘High, Small’ and the state adjective of corpus data set of <Expected result> is ‘Normal’, ‘High’ is found once but ‘small’ is not found in state adjectives of <Current state> so the multiplication of frequencies is zero, and the frequency for state adjective of <Expected result> is zero. Thus, the similarity index assessment value is zero.

As described above, by using a mechanism of multiplying frequencies, in case that all elements are found, it can be used as an additional point to the similarity index assessment value.

In addition, as shown in (b) of FIG. 4, if corpus data set has a biological word, the similarity assessing unit 124 may further generate a 1×n sub similarity matrix.

For example, in case that corpus data set of <Current state> is [blood, alcohol, level, very, high] and corpus data set of <Expected result> is [blood, alcohol, level, normal], token such as ‘blood’ is a biological word registered in the biological word dictionary. The similarity assessing unit 124 may compare the biological word to each of n biological system informations stored in the causal model database 122. If ‘blood’ was found twice in indexed terms corresponding to Organ, Part and Entity elements of j^{th} biological system information, the sum of frequencies is two, and two as the similarity index assessment value between j^{th} biological system information and token as the biological word included in corpus data set may be registered as j^{th} element of the sub similarity matrix.

As described above, the similarity assessing unit 124 may generate each of the similarity matrix and the sub similarity matrix by use of corpus data set corresponding to the retrieval query and biological system information for each organism stored in the causal model database 122. The similarity matrix is generated for all of designer's retrieval requests, but the sub similarity matrix is generated only when a biological word is included in corpus data set.

Hereinafter, a process will be described that when the similarity assessing unit 124 provides the causal model canvas unit 158 with at lease one biological system information with reference to the similarity index assessment value generated in the aforementioned process, the causal model canvas unit 158 may measure derivativity between each biological system information, and plot the network graph (See FIG. 5). Of course, it will be appreciated that the similarity assessing unit 124 may measure the derivativity and the causal model canvas unit 158 may generate the network graph by use of the result of derivativity measurement.

After assessing similarity between corpus data set and biological system information of each organism by use of the similarity matrix and/or the sub similarity matrix, the similarity assessing unit 124 may provide the causal model canvas unit 158 with at least one biological system information of which similarity index assessment value is equal to or greater than a threshold value. The threshold value may be, for example, designated as 0.75, which means to provide biological system information corresponding to the upper 75%.

FIG. 5 illustrates the network graph that the causal model canvas unit 158 generates graphically by measuring derivativity of at least one biological system information provided from the similarity assessing unit 124.

Referring to FIG. 5, a graph display screen may be divided into a graph region 510 and an information display region 520.

In the graph region 510, a network graph for biological system information assessed as having high similarity index assessment value is displayed, and numbers in series 530 for allowing a designer to select biological system information in the order of high similarity index assessment values may be disposed in upper region. If the designer changes number 1 to number 2, a network graph for biological system information in group 2 of which similarity index assessment value is relatively low may be displayed in the graph region 510.

For example, as shown in FIG. 5, a network graph corresponding to a group selected by the designer among numbers may be displayed relatively clearly in the graph region 510, but network graphs corresponding to groups not selected may be displayed relatively blurredly in the graph region 510. The designer will be able to expect existences of each network graph corresponding each number with reference to the clear network graph and the blurred network graphs.

In the graph region 510, at least one thumbnail image corresponding to other biological system information indexed with similar information for each element of biological system information may be displayed. That is, the thumbnail image may correspond to other biological system information having similar information for each element of biological system information displayed in the graph region 510, and have a hyperlink to move to biological system information of the organism when the designer selects any one of thumbnail images.

For example, if biological system information displayed in the graph region 510 relates to a Cockchafer Beetle, three thumbnail images displayed along Entity element indexed with [Melolontha, Cockchafer Beetle] relate to other three biological system information of which Entity element is indexed with information similar to Cockchafer Beetle.

In the information display region 520, biological system information that is displayed as network graph in the graph region 510 and/or related BS documents are displayed in text.

Hereinafter, a method that the causal model canvas unit 158 measures derivativity for each element of biological system information to further display thumbnail image on the network graph will be described.

The causal model canvas unit 158 may use 1×n similarity matrix in order to measure deriviativity with other biological system information by use of information indexed to each element of any one of biological system information.

The similarity matrix for measuring derivativity has a form similar to the similarity matrix described with reference to FIG. 4 (a), but information to be compared is information indexed to each element of any one of biological system information instead of corpus data set. Thus, in case that same biological system information as index information of element to be compared is compared, the similarity index will be one so this biological system information needs to be excluded from being displayed as thumbnail image.

A scheme of measuring derivativity for CoS element is shown in Expression 10.

Adj:{Adj|Adjₚᵣₑ+Adjₚₒₛₜ}

t∈S(tᵢ,tⱼ)

sim(tᵢ,tⱼ)=max[−log(p(t))]

if, a=b, then, Boolean(a,b)=1

if, a≠b, then, Boolean(a,b)=0

Scoreⱼ=sim(Nounₚᵣₑ,Nounₚᵣₑ_{ⱼ})+sim(Nounₚₒₛₜ,Nounₚₒₛₜ_{ⱼ})+Boolean(Adj,Adjⱼ) Expression 10

CoS element may be indexed, for example, as <pre condition>+<post condition> such as <Given+Olfactory+Stimulation>+<Peripheral+Sensory+Input>, and after being reconfigured as adjective set [given, peripheral], noun set of pre condition [olfactory, stimulation], and noun set of post condition [sensory, input], may be compared to index information of CoS element of other biological system information.

In comparison of adjective sets, 1 is outputted if they are matched to each other and 0 is outputted if they are not matched to each other. In the same manner as the comparison of PPH as described above, Noun sets of pre condition and post condition may be calculated in the semantic distance calculation scheme based on the energy term dictionary, the signal term dictionary and/or the material term dictionary, and then the similarity index assessment value may be calculated by summing all of these values.

A scheme of measuring derivativity for PPH element is shown in Expression 11.

t∈S{tᵢ,tⱼ}

sim(tᵢ,tⱼ)=max[−log(p(t))]

Scoreⱼ=sim(Predicate_{physical},Predicate_{physical}_{ⱼ})·sim(Object_{physical}, Object_{physical}_{ⱼ}) Expression 11

PPH element may be indexed, for example, as <verb part>+<noun part> such as <Expand>+<Surface>, and when comparing to index term of PPH element of other biological system information, the verb part may be calculated in the semantic distance calculation scheme based on the function term dictionary, and the noun part may be calculated in the semantic distance calculation scheme based on the energy term dictionary, the signal term dictionary and/or the material term dictionary. Each calculation value may be summed to be the similarity index assessment value.

A scheme of measuring derivativity for PEF element is shown in Expression 12.

if, a=b, then, Boolean(a,b)=1

if, a≠b, then, Boolean(a,b)=0

Scoreⱼ=Boolean(PEF,PEFⱼ) Expression 12

PEF element may be indexed with term such as <Surface-to-Volume Ratio> included in the PEF index term dictionary, and be compared to index term of PEF element of other biological system information. If they are matched to each other, one is outputted, and if they are not matched to each other, zero is outputted. These values may be used as similarity index assessment value.

A scheme of measuring derivativity for Input element is shown in Expression 13.

t∈s(tᵢ,tⱼ)

sin(tᵢ,tⱼ)=max [−log(P(t))]

Scoreⱼ=sim(Indexₘₐₜₑᵣᵢₐₗ,Indexₘₐₜₑᵣᵢₐₗ_{ⱼ})+sim(Index_{energy},Index_{energy}_{ⱼ})+sim(Index_{signal},Index_{signal}_{ⱼ}) Expression 13

Input element may be indexed with term such as <Olfactory Signal> included in the energy term dictionary, the signal term dictionary or the material term dictionary, and when compared to index term of PPH element of other biological system information, if indexed information such as <Olfactory Signal> corresponds to the signal index term only, the assessment results for the material index term and the signal index term may be outputted as zero. But the similarity index assessment value for the signal index term may be calculated by use of the semantic distance calculation scheme based on the signal term dictionary.

A scheme of measuring derivativity for EPH element is shown in Expression 14.

t∈s(tᵢ,tⱼ)

sim(tᵢ,tⱼ)=max[−log(p(t))]

Scoreⱼ=sim(Predicate_{ecological},Predicate_{ecological}_{ⱼ})·sim(Object_{ecological},Object_{ecological}_{ⱼ}), Expression 14

EPH element may consist of <verb part> and <noun part> such as <Locate>+<Food>, and when comparing to index term of EPH element of other biological system information, verb part may be calculated by use of the semantic distance calculation scheme based on the function term dictionary, and noun part may be calculated by use of the semantic distance calculation scheme based on the energy term dictionary, the signal term dictionary and/or the material term dictionary. Calculation values may be summed to be the similarity index assessment value.

A scheme of measuring derivativity for EBH element is shown in Expression 15.

if, a=b, then, Boolean(a,b)=1

if, a≠b, then, Boolean(a,b)=0

Scoreⱼ=Boolean(Index_{ecologicaleffect},Index_{ecologicaleffect}_{ⱼ}) Expression 15

EBH element may be indexed with term such as <Foraging> included in the EBH term dictionary, and compared to index term of EBH element of other biological system information. If they are match to each other, one is outputted, and if they are not matched to each other, zero is outputted. These values may be used as the similarity index assessment value.

A scheme of measuring derivativity for each of Organ element and Part element is shown in Expression 16.

if, a=b, then, Boolean(a,b)=1

if, a≠b, then, Boolean(a,b)=0

Scoreⱼ=Boolean(String_{organ},String_{organ}_{ⱼ}) Expression 16

Organ element and Part element may be indexed with term such as <Fan-like End>, <Antennae> included in the biological term dictionary, and compared to index term of Organ element or Part element of other biological system information. If they are match to each other, one is outputted, and if they are not matched to each other, zero is outputted. These values may be used as the similarity index assessment value.

A scheme of measuring derivativity for Entity element is shown in Expression 17.

t∈s(tᵢ,tⱼ)

sim(tᵢ,tⱼ)=max[−log(p(t))]

Scoreⱼ=sim(ID_{ITIS},ID_{ITIS}_{ⱼ}) Expression 17

Entity element may be indexed by including ITIS unique ID (i.e., numeral code for scientific name designated by International standard ITIS), and the similarity index with unique ID of Entity element of other biological system information may be calculated in the same manner of calculating the semantic distance based on a hierarchical tree data structure that unique ID of scientific name has.

A scheme of measuring derivativity for Action element is shown in Expression 18.

if, a=b, then, Boolean(a,b)=1

if, a≠b, then, Boolean(a,b)=0

Scoreⱼ=Boolean(String_{action},String_{action}_{ⱼ}) Expression 18

Action element may be indexed with a combination of words such as <Maximize Exposure>, and compared to index term of Action element of other biological system information. If they are matched to each other, one is outputted, and if they are not matched to each other, zero is outputted. These values may be used as similarity index assessment value.

By aforementioned schemes, the derivativity for each element of biological system information of which network graph is displayed in the graph region 510 may be calculated, thumbnail images for a predetermined number of other biological system information having high derivativity for each element may be displayed to correspond to each element of biological system information constituting the network graph.

As described above, the biological system information retrieval system according to the present embodiment may implement biological system information, which is subject of mimicking and applying in bio-inspired design, including physical relations, ecological relations and/or biological relations, as a comprehensive causal model, and construct it by ontology. Thus, it is advantageous for a designer in the bio-inspired design to conduct an effective retrieval by using various information and conditions, and to help the designer design inventively.

According to another embodiment of the present invention, an apparatus and method for reasoning biological information using the species identification may be provided. The biological information reasoning device and method can help to accurately capture the external or ecological characteristics of each organism by using the species identification key in the field of biology.

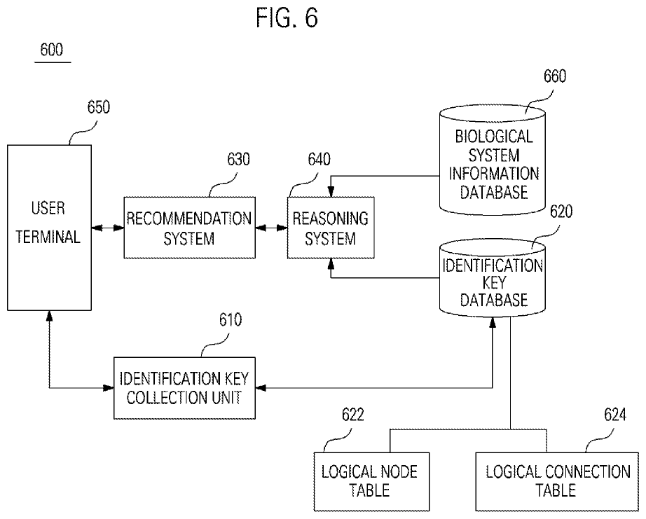

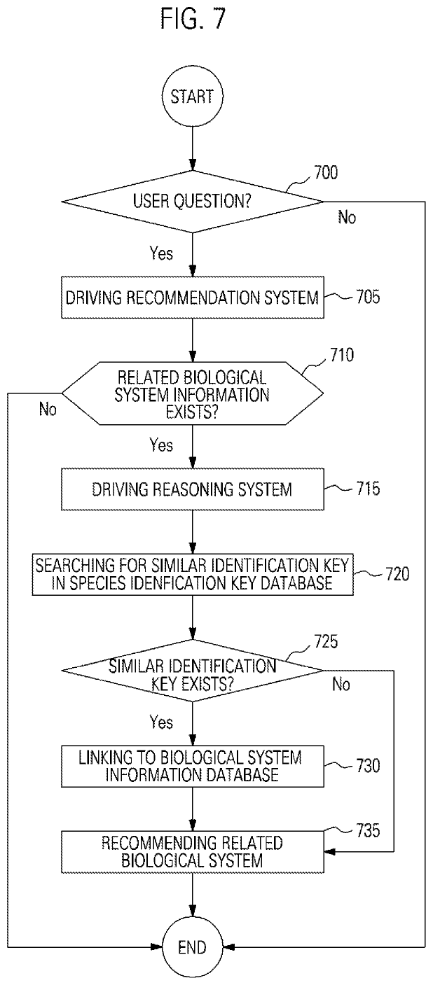

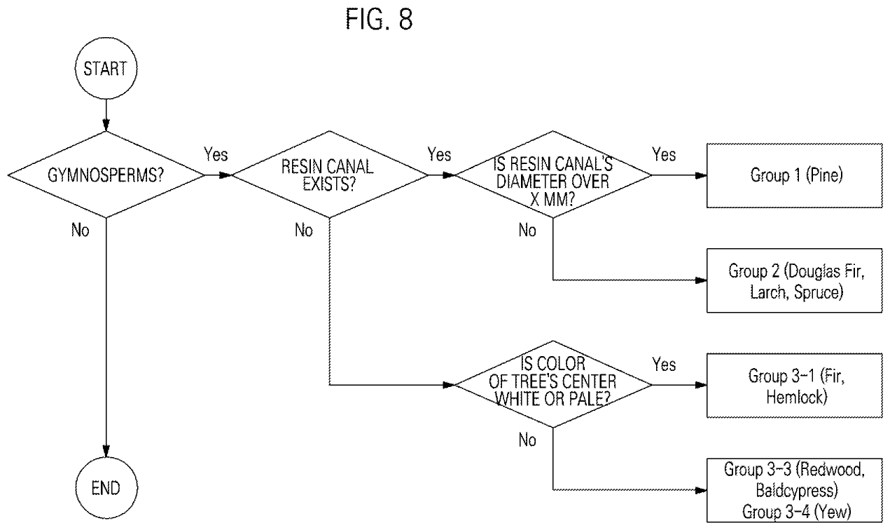

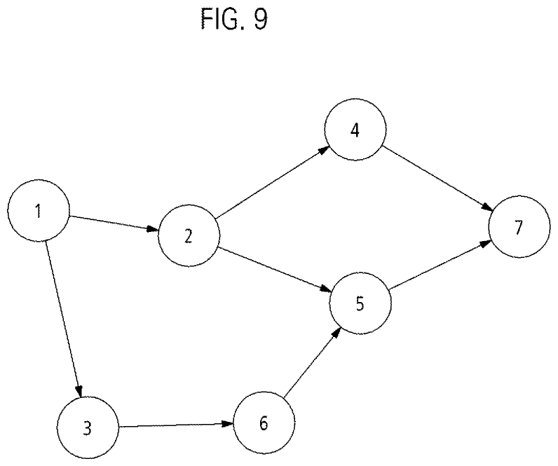

FIG. 6 is a block diagram of an apparatus of reasoning biological system characteristics through species identification according to another embodiment of the present invention, FIG. 7 is a flowchart of a method of reasoning biological system characteristics through species identification according to another embodiment of the present invention, FIG. 8 is a classification diagram showing the biological species of soft trees, and FIG. 9 is an exemplary diagram of a DAG graph.

Species identification refers to the act of revealing the scientific name and common name of an organism to which taxon it belongs. In other words, it refers to the act of clarifying the collected unknown organism is. Identifying the fossils found or the pollen collected is also called as identification.

From the academic point of view, the species identification key in the current biology field is being used to accurately identify the scientific names (‘species’ and ‘genus’ names) of observed organisms in nature, and objectively (scientifically) clearly identify what kind of organism the discovered organism is.

From the industrial aspect, it is used to accurately use naturally derived substances without confusion. This is a very important issue in the food and pharmaceutical industries that are directly related to human health. This is because the wrong use of living organisms in the food and pharmaceutical industries that use naturally derived substances may provide a source of risk. For example, the species identification is used to avoid misuse of organisms that may be confused because of their similar appearance.

In addition, for other academic or industrial purposes, the species identification is being used in an imitated form in other academic or industrial fields. Identification similar to the biological species identification is being used to specify a disease, to specify the type of soil, to specify the type of ore, or to specify the age of archaeological or anthropological relics.

The species identification key is a set of questions used to identify the biological species. In general, the species identification key is a set of questions with a hierarchical structure.

The questions used for identification are called ‘identification keys’. By answering the identification key, it will be possible to identify the organism. For example, if you want to specify what kind of shrimp the discovered shrimp is, you can answer the ‘identification key’ with a set of species identification keys for shrimp (the number of the identification key for shrimp is 148).

The identification key was generally used in the form of step-by-step questions of a tree structure. However, recent advances in genetics and molecular biology have made it possible to identify species more accurately, and thus the species identification key has become more complicated.

Recently, beyond the general tree structure, there is a trend that the identification key set is also transformed to have a DAG (Directed Acyclic Graph) form with a direction. The DAG is a graph that has a direction, but does not have a circular structure in the graph.

As shown in FIG. 9, nodes 6, 5, and 7 are connected to each other with directionality, but there is no cyclic connection (for example, 6->5->7->6). Therefore, although the structure is more complicated than the decision graph of the old tree structure, it has an acyclic structure so that one specific organism can be specified in the end (a tree-structured decision graph is a type of DAG graph).

Therefore, by answering a series of questions (identification keys), it becomes finally possible to specify the type of organism (like twenty questions, according to the answers to the questions, the possible types of living organism can be narrowed), which is the scientific basis for making the researchers to specify the name of the observed organism. Therefore, these questions (species identification keys) can be said to contain essential elements necessary to clearly define the identity of a specific creature. Therefore, the species identification keys that are standardized with the consent of the biological community are being used in academia.

For example, assuming a situation to analyze the exact scientific name of ‘ant’ observed in nature, researchers must answer the following identification key questions. By answering questions such as the shape of the ant's head, the presence or absence of hair on a specific body part, the color of a specific body part, and whether there are more than 12 tactile nodes or less, it is possible to specify the type of ‘ant’ in the end.

As defined by the “National Institute of Biological Resources” (https://species.nibr.go.kr/UPLOAD_TOTL//CMS/342/content.htm;jsessionid=Vu01qAw1jaaR12HSYMz11XLd4yQtJhvm42WqfNRia0nH2PfmyQYPWQ9PqDDZwwla.totl_was_servlet_engine1), the species identification key are being utilized for the purpose of (1) quality assurance of biological resources (prevention of misuse of similar biological species by biologically derived substances such as herbal medicines), (2) use of biological resources (purpose of the ecological survey, such as planned development, to conduct an ecological survey without any mistake), and (3) clear distinction between native organisms and ecosystem-destroying species. In the ecological survey, it is very important to know the exact scientific name because the number of species living in the area is determined by the method of finding out what kinds of organisms are living in a specific area. In particular, the Delta Project, which developed IntKey (Species Identification System) in the US, also states that it was developed for the purpose of effective classification of biological resources (https://www.delta-intkey.com/www/overview.htm). Therefore, the species identification key is being used only for the purpose of efficient management of species resources.

However, since the species identification key contains clear rationale factors that can differentiate species A and species B, it can be used very efficiently to conversely infer the unique characteristic elements of each species. For example, if we look back through the example to clearly classify the species of ‘ant’, the following scenario is possible. If one species of the ants with ‘12 or more tactile nodes’ has been found to have special abilities such as location tracking and individual tracking, other ant species with ‘12 or more tactile nodes’ can be inferred to have similar abilities.

Alternatively, self-healing ability of a spruce is attracting attention, and in the species identification of soft trees, species are classified by the presence or absence of “resin canal” (see FIG. 8).

It can be inferred from this that Groups 1 and 2 are both biological systems with self-healing ability (spruce belongs to Group 2). Because it is resin that plays a key role in self-healing ability, it can be inferred that similar species with a ‘resin canal’ that emits resin all have self-healing ability.

That is, when information from other existing biological systems and information on the species identification key are combined, the computer system can reason new facts from relationships that we are not aware of.

However, the current method of using the species identification key and the current information system of biological system did not propose a scientific method of using the species identification key, and neither the algorithm nor the information exchange device was proposed or developed.

The apparatus 600 of reasoning biological system information utilizing the species identification of the present invention may comprise an identification key collection unit 610 configured for collecting biological identification keys consisting of DAG graphs, an identification key database 620 configured for hierarchically managing a question set of the collected species identification keys, a reasoning system 640 configured for reasoning characteristics of the biological system from the question set of the identification keys and the information system of biological system, a biological system recommendation system 630 configured for recommending a biological species (=biological system) according to user's questions, and a user terminal 650 configured for helping a user interact with the storage and systems (see FIG. 6).

The identification key collection unit 610 collects the species identification keys composed of DAG graphs.

The species identification key has not yet been unified internationally. That is, unlike the industrial standards unified by international certification, the set of identification keys widely used in academia by biological group is regarded as a standard and is commonly used. An identification key repository or database for all biological species has not yet been completed.

In addition, the scale of the identification key set is not unified for each biological group. Therefore, they are bound to be different. Some types of identification key sets are for an entire ‘Order’, and some types of identification key sets are limited to specific ‘Genus’.

For example, for the identification of ‘Macrura’, the set of identification keys for Dendrobranchiata ‘suborder’ under Decapoda ‘order’ can be used. The identification keys for identifying all species belonging to one Dendrobranchiata ‘suborder’ is provided.

On the other hand, in the case of plant species identification, set of identification keys for several orders rather than identification keys for one order is used. As shown in the figure of ‘softwood’ species identification in FIG. 8, the species identification key set for Gymnosperms (softwood) provides comprehensive identification keys regarding several plant orders.

For each biological group, the set of identification keys in scale suitable for classification is used. In the case of ‘Macrura’, unlike plants, a more limited set of identification keys can be used than for plants because the characteristics can be easily captured with the eye and classified as ‘Macrura’. Specifically, ‘Macrura’ is phylogenetically branched from ‘Anomura’ and ‘Brachyura’ and the order Decapoda. Since shrimp's external characteristics can be clearly distinguished from ‘Anomura’ and ‘Brachyura’, the identification key of shrimp does not necessarily consist of the identification key for order Decapoda group, but rather consists of a limited identification key for a more detailed suborder of ‘Macrura’ group.

Each identification key set consists of a set of several questions as described above. It has the DAG structure, starts with the topmost question, and moves on to the next question with a direction depending on the answer. By answering the chained questions, one specific species can be finally specified.

However, since the identification key sets have not yet been internationally standardized, many of them have not yet been converted into a database, and only some are provided in the form of databases. The identification for most living organisms is still using the printed identification key sets that has been handed down in each research field.

For this reason, most species identification systems (namely, computer systems), only the system is provided so that the user can directly input the identification key used for each species and store it online for use. There is a limitation that the key cannot be provided.

Therefore, it is necessary to specify the method and system of collecting and databasing the species identification keys.

The species identification key collection unit 610 may store the identification key sets existing as a document (including a printed document) in a database. This may be implemented through the user terminal 650 having a user interface.

Basically, the components of the species identification key set are as follows.

(1) “Logical node”, which is a ‘logical question’ that can be answered with yes or no;

(2) A chain of “logical connections (yes or no connections)” of questions (logical nodes);

(3) One “starting point” (one topmost question (logical node)); and

(4) Multiple “endpoints” (species specified through a chain of answers to logical questions).

In constructing the species identification key set, it is necessary to satisfy the following conditions.

(a) Only one yes or no “logical connection” can be derived from each “logical node”

(b) A “logical node” can receive connections from multiple “logical connections”

(c) A cascading connection of “logical connection” cannot be a circular connection (it must be a DAG graph)

(d) only one biological species may be assigned to one “endpoint”

(e) “start point” is one “logical node”

Since the features possessed by one organism corresponding to the “end point” can be reasoned from the “logical nodes” and “logical connections” between the corresponding end point and the starting point, the above four factors (1)˜(4) are essential.

In addition, the purpose of the present system is not to identify species, but to reason characteristics of an organism from species identification key information, so even if (2) “logical connection” is not a logical connection with a direction, but a (insufficient) connection without direction, even with a set of “logical node” texts between the “start point” and “end point”, rough content of the ‘feature’ information can be reasoned using techniques such as machine learning.

The identification key database 620 hierarchically manages the set of questions of the collected species identification key.

Basically, the database can satisfy the four essential components by having the following two types of tables and columns.

A logical node table 622 corresponding to [Table 1] includes a “logical node” unique number (column 1), “logical node” text (column 2), and a related image (column 3).

The logical connection table 624 corresponding to [Table 2] includes a corresponding species unique number (column 1) and a “logical connection” graph (column 2) of the logical node numbers.

Relevant images are assigned to the database only if there is an image that can be referenced for each ‘logical node’ question.

Species unique number can use the US ITIS (Integrated Taxonomic Information System) that provides standardized information on the largest number of species in biological lineage classification. In addition, it is possible to configure the identification key database by using several phylogenetic information systems.

If performing ‘species identification’ or other functions other than reasoning biological characteristics by using this database is required, the database can be configured in various forms other than the above.

For example, the species unique number may be linked with the scientific name and common name of the organism, and in addition, it may have a relationship with the database such as the image of the organism, the habitat of the organism, and related thesis data.

The reasoning system 640 reasons the characteristics of the biological system from the set of questions of the identification key and the information system of biological system. The biological system recommendation system 630 recommends a biological species (=biological system) according to a user's question.

Of the information stored in the identification key database 620, the reasoning system can basically reason feature information of an organism essentially with [Table 1] (column 2) “logical node” text, [Table 2] (column 1) species unique number and [Table 2] (column 2) “logical connection” graph information. These three kinds of data resources are the smallest set for reasoning.

Of course, more precise reasoning can be performed by linking with additional databases.

In the data resource described above, the “logical node” text connected by “logical connection” is a set of answers that are all yes as a result of the question of the logical node. Therefore, all words appearing in the text of the corresponding logical node indirectly describe the features of the corresponding organism.

The sentence structure of a question can be analyzed using a natural language processing technique, and biological information reasoning technique applied to the aforementioned biological system information retrieval system can be used to reason feature information of organism.

The biological system recommendation system 630 may recommend feature information of organism by using the above-described biological information reasoning technique.

The operation of the recommendation system 630 and/or the reasoning system 640 may be based on the scenario shown in FIG. 7. This scenario is an example of performing reasoning and recommendation as soon as a query is input.

Alternatively, the system can be operated in such a way that reasoning and recommendation are performed in advance, the result value is stored in other storage or database in advance, and a pre-calculated value is called whenever a query is input.

Referring to FIG. 7, the biological information reasoning method performed by the biological information reasoning apparatus according to the present embodiment may perform the following steps.

A user may question the system for design issues (700).

For example, “method of autonomously detecting objects and obstacles” can be the question.

This is the same as a searching method performed in the biological system information retrieval system described above.

The user terminal receives the corresponding question and transmits it to the recommendation system 630 (705).

A method for the recommendation system 630 to recommend the biological species (=biological system) is also the same as the recommendation method performed by the above-described biological system information retrieval system.

If the related biological system information exists (710), the reasoning system 640 is driven (715) to search a similar identification key in the species identification key database 620 (720).

It is determined whether the similar identification key exists (725), and if there is the similar identification key, it is linked to a biological system information database 660. And the related biological system is recommended (735).

The information stored in the biological system information causal model is used, and “part” and “organ” of the biological relationship have an additional connection relationship with the species identification key information. In addition, the “ecological behavior” of the ecological relationship has an additional connection with the species identification key information.

Therefore, in the biological system information retrieval system according to one embodiment, if the recommendation action was performed with only information indexed in the causal model, in this embodiment, the information of the identification key database 620 and the above three elements “part”, “organ”, and “ecological behavior” have connection relationships, which make it possible to find organisms that could not be found before.

When a relationship to be mapped with identification key information in addition to the three elements occurs later, elements other than the three elements may also establish a connection relationship with the identification key information (720).

The identity key database contains a hierarchical relationship of identification keys.

Referring to FIG. 6 again, the user terminal 650 facilitates interaction of the user with the storage and systems.

The user terminal 650 is a computing device that can access a system implemented in the form of an online website, and may be, for example, a PC, a notebook computer, a tablet PC, or a smart phone.

The user terminal 650 may input a ‘query’ so that the user may drive a recommendation function. And the user may edit the species identification key through the user terminal (650).

Although the above has been described with reference to the embodiments of the present invention, those of ordinary skill in the art can variously modify the present invention within the scope without departing from the spirit and scope of the present invention described in the claims below. and may be changed.

What is claimed is:

1. A biological information reasoning apparatus utilizing a species identification, comprising:
an identification key collection unit configured for collecting a species identification key set;
an identification key database configured for hierarchically managing a set of questions of the collected species identification key set;
a recommendation system configured for recommending a biological system according to a user's question through a user terminal; and
a reasoning system configured for reasoning characteristics of the biological system from the set of questions of the collected species identification key set regarding a species corresponding to the biological system and a pre-built information system of biological system.

2. The biological information reasoning apparatus of claim 1, wherein the species identification key set consists of DAG graphs.

3. The biological information reasoning apparatus of claim 1, wherein the species identification key set comprises
a logical node, which is a logical question that can be answered with yes or no; and
a chain of logical connections of the logical questions,
wherein the species identification key set comprises one starting point and a plurality of endpoints as the logical nodes.

4. The biological information reasoning apparatus of claim 3, wherein in the species identification key set, only one yes or no logical connection is derived from each logical node, the logical node receives connections from multiple logical connections, the chain connection of logical connections is acyclic connection, and only one biological species is assigned to one endpoint.

5. The biological information reasoning apparatus of claim 3, wherein the identification key database comprises:
a logical node table having a logical node unique number and a logical node text; and
a logical connection table having a biological species unique number and a logical connection graph of logical node numbers.

6. The biological information reasoning apparatus of claim 5, wherein the reasoning system reasons a feature information of an organism with the logical node text of the logical node table, and the species unique number and the logical connection graph of the logical connection table.

7. A biological information reasoning method being performed by a biological information reasoning apparatus utilizing a species identification, comprising:
transmitting a user question from a user terminal to a recommendation system;
analyzing, by the recommendation system, the user question;
driving a reasoning system when a relevant biological system information exists as a result of the analysis exists;
searching for, by the reasoning system, a similar identification key from an identification key database; and
linking to a biological system information database and recommending a related biological system.

8. The biological information reasoning method of claim 7, wherein in the case of linking to the biological system information database, a part and organ of the biological relationship according to a biological system information causal model of the biological system information database have an additional connection relationship with species identification key information, and an ecological behavior of the ecological relationship has an additional connection with the species identification key information.

9. The biological information reasoning method of claim 7, wherein the identification key database hierarchically manages a set of questions of the species identification key set,
wherein the species identification key set consists of DAG graphs.

10. The biological information reasoning method of claim 9, wherein the species identification key set comprises
a logical node, which is a logical question that can be answered with yes or no; and
a chain of logical connections of the logical questions,
wherein the species identification key set comprises one starting point and a plurality of endpoints as the logical nodes.

11. The biological information reasoning method of claim 10, wherein in the species identification key set, only one yes or no logical connection is derived from each logical node, the logical node receives connections from multiple logical connections, the chain connection of logical connections is acyclic connection, and only one biological species is assigned to one endpoint.

12. The biological information reasoning method of claim 11, wherein the identification key database comprises:
a logical node table having a logical node unique number and a logical node text; and
a logical connection table having a biological species unique number and a logical connection graph of logical node numbers.

13. The biological information reasoning method of claim 12, wherein the reasoning utilizes the logical node text of the logical node table and the species unique number and the logical connection graph of the logical connection table to reason a feature information of an organism.
